(19) United States
(12) Patent Application Publication
(10) Pub. No.: US 2011/0028420 A1
(43) Pub. Date: Feb. 3, 2011

(54) Regression of Established Atherosclerotic Plaques, and Treating Sudden-Onset Asthma Attacks, using PARP Inhibitors

(76) Inventors: Hamid A. Boulares, Kenner, LA (US); Chetan P. Hans, Upper Arlington, OH (US); Amarjit S. Naura, New Orleans, LA (US)

Correspondence Address: PATENT DEPARTMENT;TAYLOR, PORTER, BROOKS & PHILLIPS, L.L.P, P.O. BOX 2471, BATON ROUGE, LA, 70821-2471, US

(21) Appl. No.: 12/936,733

(22) PCT Filed: Apr. 7, 2009
(86) PCT No.: PCT/US09/39743

§ 371 Date: Oct. 7, 2010

Related U.S. Application Data
(62) Division of application No. 61/043,923, filed on Apr. 10, 2008.

(51) Int. Cl. A61K 31/4743; A61K 31/65; A61K 31/4709; A61K 31/166; A61K 31/473; A61K 31/47; A61K 31/517; A61K 31/352; A61K 31/7076; A61K 31/519; A61K 31/502; A61K 31/496; A61K 31/436; A61K 31/551; A61K 31/407; A61K 31/4184; A61P 11/06; A61P 9/10

(52) U.S. Cl. 514/46; 514/152; 514/191; 514/291; 514/619; 514/298; 514/309; 514/296; 514/266.3; 514/457; 514/260.1; 514/248; 514/253.02; 514/288; 514/220; 514/410; 514/394

(57) ABSTRACT

A method is disclosed for treating and inducing the regression of established atherosclerotic plaques. A method is disclosed for treating asthma, including treatment of an ongoing asthma attack. In both cases, treatment with PARP inhibitors, such as the PARP inhibitor TIQ-A (Thieno[2,3-c]isoquinolin-5-one), can lead to regression of existing disease and symptoms.

The development of this invention was partially funded by the United States Government under grants 1P20RR8766 and HL072889 awarded by the National Institutes of Health. The United States Government has certain rights in this invention.

The benefit of the 10 Apr. 2008 filing date of United States provisional patent application serial number U.S. 61/043,923 is claimed under 35 U.S.C. §119(e) in the United States, and is claimed under applicable treaties and conventions in all countries.

### TECHNICAL FIELD

This invention pertains to methods for causing the regression of established atherosclerotic plaques, and for treating sudden-onset asthma attacks.

### BACKGROUND ART

Atherosclerosis

Atherosclerosis is a major cause of morbidity and mortality in developed countries. It is the underlying cause of several cardiovascular diseases. Many factors contribute to atherosclerosis. Inflammation has increasingly been recognized as playing a major role in atherosclerosis. Risk factors such as dyslipidemia also contribute.

Atherosclerosis is a slowly developing, progressive disease. Lipids and fibrous components accumulate in the intima, the innermost layer of medium and large arteries. Arterial fatty streaks form as lipids accumulate within macrophages and smooth muscle cells (SMC). The early stages of the disease, characterized by intracellular accumulation of lipids in phagocytes, are clinically “silent.” As macrophages accumulate lipoproteins, some differentiate into foam cells. As the immune system attempts to clear these fatty streaks, chronic inflammation develops at the site of the lesion. Inflammation creates conditions that promote plaque development and progression. When a plaque ruptures, cardiovascular disease results, with symptoms such as thrombotic occlusion of arteries, which can lead to stroke or heart attack.

Most prior treatments have focused on stabilizing vulnerable plaques, for example by balloon angioplasty. It would be highly desirable to have an effective pharmaceutical treatment to induce the regression of existing atherosclerotic plaques. However, there are no drugs or other treatments currently available to induce regression of existing plaques. Instead, symptoms of atherosclerosis are typically “managed” by the control of risk factors such as dyslipidemia, hypertension, and smoking. There remains a critical, unfilled need for therapeutic interventions that can cause the regression of established atherosclerotic lesions.

Promoting the regression of plaques is a very different problem from inhibiting plaque formation. A number of compounds have been reported in the art to inhibit the formation of plaques. But few, if any, of these compounds have been reliably reported to induce the regression of existing plaques.

Poly(ADP-ribose) polymerase (PARP) plays an important role in tissue injuries that are associated with oxidative stress or inflammation. PARP activation catalyzes the covalent coupling of branched chains of ADP-ribose units to various nuclear proteins, such as to histones and to PARP itself. Excessive PARP activation contributes to inflammation, and depletes cellular stores of NAD and precursor ATP. The depletion of NAD and ATP can lead to irreversible cytotoxicity and cell death. PARP affects NAD metabolism, and thereby also affects cell and tissue homeostasis. PARP is believed to regulate the expression of several inflammatory factors, including adhesion molecules, TNF, interleukins, and inducible nitric oxide synthase (iNOS). We and our colleagues recently reported that poly-ADP-ribose polymerase (PARP) is activated within atherosclerotic plaques in an animal model of atherosclerosis. See K. Oumona-Benachour et al., Circulation, vol. 115, pp. 2442-2450 (2007).

T. von Lukowicz et al., “PARP1 is required for adhesion molecule expression in atherogenesis,” Cardiovascular Res., vol. 78, pp. 158-166 (2008) reported experimental results in mice supporting a conclusion that PARP1 increased expression of adhesion molecules, promoted plaque inflammation, and induced features of plaque vulnerability. The authors concluded that pharmacological inhibition of PARP via chronic administration of PJ34 (a PARP inhibitor), or via knock-out of the PARP1 gene, markedly decreased plaque formation in atherosclerosis.

Asthma

It is generally accepted that airway inflammation is a key factor underlying asthma. Asthma involves both cellular and non-cellular factors. Antigen exposure initiates a cascade that leads to the generation of CD4+ Th2 cells, IgE secretion, recruitment of eosinophils into the airways, airway hyperresponsiveness, hyperplasia of goblet cells, and over-secretion of mucus. CD4+ Th2 cells play a major role in the allergic response, including their release of key cytokines that promote the build-up of eosinophils in the lungs and that stimulate B lymphocytes to secrete IgE.

There is an unfilled need for improved treatments for asthma, particularly for treatments that will work rapidly when administered after the onset of an acute asthma attack. Corticosteroids are currently the most widely used anti-inflammatory treatment for asthma. But their action is non-specific, and there are concerns about side effects and compliance issues, particularly in children and adolescents.

Several strategies that have shown potential when tested in experimental animal models of asthma have encountered limitations when used to treat humans—limitations of efficacy, cost, or both. Existing asthma preventatives typically require weeks before efficacy begins. There is an unfilled need for effective treatments for asthma, especially for treatments that will act quickly when administered following acute allergen exposure.

Another approach, that has shown partial success, has been to block either the release or the effects of Th2 cytokines. Inhibition of IL-5 with monoclonal antibodies has been effective in reducing circulating and airway eosinophils, but inhibiting IL-5 does not appear to have much effect on a subject's clinical symptoms. IL-4-blocking antibodies have inhibited allergen-induced airway hyper-responsiveness (AHR), goblet cell metaplasia, and pulmonary eosinophilia in a murine model, but were disappointing when tested clinically. Antibodies against IL-13 have been effective in reversing chronic allergic inflammation; however IL-4 can induce similar acute changes in the airways, even in the absence of IL-13 signaling, including BAL fluid eosinophilia, AHR, and goblet cell hyperplasia. Thus blocking IL-13 appears to have only modest clinical benefits.

Other studies have explored cytokine receptors, chemokine receptors, or small molecule receptor antagonists as asthma therapies. Still other approaches have attempted to block the signal transduction pathways that are activated when cytokines or chemokines interact with their receptors.

Airway eosinophilia is consistently observed in allergic inflammation and asthma. Activated eosinophils release lipid mediators, cytokines, and cytotoxic proteins. Despite the clear association between eosinophil numbers and the severity of asthma symptoms, the actual role of the leukocytes in asthma remains elusive. There have been recent reports that asthma could not be experimentally induced in transgenic animals that are deficient in eosinophils, a finding that strongly supports the conclusion that eosinophilia is integral to asthma.

P. Barnes, “New therapies for asthma,” Trends Mol. Med., vol. 12, pp. 515-520 (206) reported that blocking the production of IL-13, for example by administering soluble IL-13Ra2-Ig, or by IL-13 gene deletion, inhibits airway production of mucus in mice.

A. Daoud et al., “Minocycline treatment results in reduced oral steroid requirements in adult asthma,” Allergy and Asthma Proceedings, vol. 29, pp. 286-294 (2008) reported results of a small trial in which twice-daily administration of minocycline to asthma patients for eight weeks was associated with a 30% reduction in mean daily prednisone use versus placebo.

Minocycline and other tetracycline derivatives have been reported to have neuroprotective effects unrelated to their antimicrobial properties. C. Alano et al., “Minocycline inhibits poly(ADP-ribose) polymerase-1 at nanomolar concentrations,” PNAS, vol. 103, pp. 9685-9690 (2006) report in vitro studies using cortical neuron cultures in which induced PARP1 activation was observed to be inhibited by either 3,4-dihydro-5-[4-(1-piperidinyl) butoxy]-1(2H)-isoquinolinone, a previously known PARP inhibitor, or by minocycline. Other tetracycline derivatives showed similar qualitative effects, with the rank order of potency being minocycline>doxycycline>demeclocycline>chlortetracycline.

R. DiPaola et al., “Treatment with PARP1 inhibitors, GPI 15427 or GPI 16539, ameliorates intestinal damage in rat models of colitis and shock,” Eur. J. Pharm., vol. 527, pp. 163-171 (2005) reported that post-injury administration of the PARP1 inhibitors GPI 15427 or GPI 16539 exerted potent anti-inflammatory effects in a rat model of gut inflammation and inflammation. The authors also reported that GPI 15427 or GPI 16539 treatment diminished the accumulation of poly (ADP-ribose) in the ileum of splanchnic artery occlusion-shocked rats and in the colons of dinitrobenzene sulfonic acid-treated rats; and concluded that these two compounds might be useful for treating gut ischemia and inflammation.

Recent work by us and our colleagues reported that PARP plays a role in eosinophil recruitment into lungs in an experimental model of allergic airway inflammation. M. Oumona et al., “PARP1 inhibition prevents eosinophil recruitment by modulating Th2 cytokines in a murine model of allergic airway inflammation: a potential specific effect on IL-5,” J. Immunol., vol. 177, pp. 6489-6496 (2006). Administration of the PARP inhibitor thieno[2,3-c]isoquinolin-5-one (TIQ-A) prior to allergen challenge suppressed Th2 cytokines, including IL-4 IL-5 and IL-13, and suppressed eosinophil infiltration and associated mucus production in lung airways.

At least 18 enzymes within the PARP family have been reported to date, of which the 113 kDa PARP1 is the most abundant, accounting for perhaps 85% of overall cellular PARP activity. The PARP enzymes share the capacity to polymerize ADP-ribose from NAD (nicotinamide adenine dinucleotide). Due to the conserved NAD+ binding site used by the PARPs, and due to the high levels of sequence homology or structural homology among the catalytic domains of the PARP family of enzymes, these enzymes are frequently blocked by the same inhibitors. Most PARP inhibitors will inhibit most, if not all, enzymes within the PARP family, although the relative degree of inhibition may change from one to another. For example, the inhibitor 5-benzoyloxyisoquinoline has a 60-fold selectivity for the PARP-2 isotype over the PARP-1 isotype, although it does inhibit both isotypes. Also, PARP-1 is the predominant form present in cells, typically with a 10- to 15-fold higher activity than PARP-2. Thus PARP-1 is normally the primary target for PARP inhibitors, although most PARP-1 inhibitors will also inhibit other PARP isotypes, and vice versa. See generally J. Amé et al., “The PARP Superfamily,” BioEssays, vol. 26, pp. 882-893 (2004); and S. Smith, “The world according to PARP,” Trends Biochem. Sci., vol. 26, pp. 174-179 (2001).

K. Ratnam et al., “Current Development of Clinical Inhibitors of Poly(ADP-Ribose) Polymerase in Oncology,” Clin. Cancer Res., vol. 13, pp. 1383-1388 (2007) reviews the literature on PARP, PARP inhibitors, and their use in cancer therapy. See also N. Curtin, “PARP inhibitors for cancer therapy,” Expert Reviews in Molecular Medicine, vol. 7:4, pp. 1-20 (2005).

P. Pacher et al., “Role of Poly(ADP-ribose) polymerase 1 (PARP-1) in Cardiovascular Diseases: The Therapeutic Potential of PARP Inhibitors,” Cardiovascular Drug Reviews, vol. 25, pp. 235-260 (2007) reviews the literature on PARP, PARP inhibitors, and their effects in myocardial ischemia/reperfusion injury, various forms of heart failure, cardiomyopathies, circulatory shock, cardiovascular aging, diabetic cardiovascular complications, myocardial hypertrophy, atherosclerosis, vascular remodeling following injury, and angiogenesis.

Alexis Biochemicals, “The PARP Family & ADP Ribosylation,” Product Flyer (dated May 15, 2008, downloaded from www.axxora.com) discloses a number of commercially available PARP enzymes, their activities, their inhibitors, antibodies, activators, and related compounds.

One cannot reliably extrapolate from observations concerning reduction in the onset of a disease to a prediction that the same agents will promote regression of existing disease. The mechanisms that are involved in inhibiting the onset of disease, versus regression of existing disease will generally differ, and in the particular cases of atherosclerosis and asthma they almost certainly are different. That this is the case may be inferred, for example, from the observation that hundreds or perhaps thousands of compounds have been reported in the literature as inhibiting the onset of atherosclerotic plaques and asthma. Yet there are few, if any, compounds that have been reliably reported to cause regression of existing disease in either case, and none that have been successful for clinical use.

For example, statins are perhaps the leading class of pharmaceuticals used to control blood cholesterol and lipid levels. However, none of the statins cause regression of existing plaques when administered at ordinary dosage levels. There has been at least one report that statins may cause some regression when administered at high dosages, but that report remains controversial.

There is an unfilled need for compounds that can be used to treat and cause regression of existing disease in atherosclerosis and asthma.

### DISCLOSURE OF INVENTION

We have discovered a method for treating and inducing the regression of established atherosclerotic plaques. We have discovered a method for treating asthma, including treatment of an ongoing, sudden-onset asthma attack. In both cases, treatment with PARP inhibitors, such as the PARP inhibitor TIQ-A (Thieno[2,3-c]isoquinolin-5-one), can lead to regression of existing disease and symptoms.

Although we have previously reported that TIQ-A can inhibit the progression of plaques, it was nevertheless surprising to discover that PARP inhibition by TIQ-A can also induce regression of plaques. To our knowledge, no other anti-inflammatory agent has previously been reported to have such an effect. The mechanism by which PARP inhibitors cause regression of plaques is unknown, although the data reported below suggest some possibilities. For example, it appears that regression is associated with an increase in collagen and smooth muscle cell content, and a decrease in macrophage recruitment and inflammatory factors such as MCP-1, ICAM-1, and TNF. The mechanism, whatever it should turn out to be, presumably differs from the mechanism for inhibiting progression of disease.

Existing asthma preventatives typically require weeks or months to take effect. It is surprising that administering a PARP inhibitor such as TIQ-A is effective against an acute (sudden-onset) asthma attack within minutes, when administered as a single dose after acute allergen exposure. The PARP inhibitor may be administered after the onset of an asthma attack, preferably within 0 to 12 hours, more preferably within 0 to 6 hours, and most preferably within 0 to 1 hour of the beginning of a sudden-onset asthma attack. It thus may not be necessary for asthmatic patients to take the PARP inhibitor over long periods of time as a preventative; instead, the PARP inhibitor could be taken only as needed to reverse an ongoing, acute asthma attack. PARP inhibitors act to inhibit the production of key asthma-inducing cytokines, including IL-5, IL-10, and GM-CSF. They inhibit the infiltration of eosinophils and macrophages into allergen-exposed lungs. PARP inhibitors appear to target the source of the inflammatory response to allergens, not just asthma's symptoms.

Following an asthma attack, it is imperative to restore normal breathing quickly. Bronchodilators such as β-agonists are commonly used to restore normal breathing at the onset of an asthma attack. While bronchodilators can help to open airways, they do not affect the underlying inflammation. Additional therapeutic means are needed to control the disease. Surprisingly, we found that PARP inhibition by TIQ-A not only ameliorated inflammation, but also restored normal breathing as assessed by reaction to methacholine (a broncho-constrictor) following allergen exposure. PARP inhibitors may thus be used both for prevention and as a rescue drug in asthmatic patients; this dual effectiveness is unique and is most surprising. We have shown that PARP inhibition, for example by TIQ-A, will protect against allergen-induced airway eosinophilia, mucus production, and airway hyperresponsiveness when administered after allergen exposure.

In addition to TIQ-A, we have also obtained promising preliminary results with the PARP inhibitors 5-aminoisoquinolinone(AIQ), 3-Aminobenzamide (3-AB), and PJ-34. Other PARP inhibitors may also be used in practicing this invention. (3-AB has a somewhat different mode of action.) For example, the following PARP inhibitors are commercially available from Alexis Biochemicals (www.axxora.com; data sheets for each compound are available through the same website): 1,5-Isoquinolinediol; 3-Methyl-5-AIQ hydrochloride; 4-Amino-1,8-naphthalimide; 4-Hydroxyquinazoline; 5-AIQ hydrochloride; 5-Iodo-6-amino-1,2-benzopyrone; 6(5H)-Phenanthridinone; EB-47 dihydrochloride dihydrate; NU1025; TIQ-A; DR2313; PJ-34.

PARP inhibitors and their sources also include: BSI 401 (BiPar Sciences); BSI 201 (BiPar Sciences); AZD 2281 (KU-0059436) (KuDOS Pharmaceuticals); INO 1001 (Inotek Pharmaceuticals); GPI 15427 (10-(4-methyl-piperazin-1-ylmethyl)-2H-7-oxa-1,2-diaza-benzo[de]anthracen-3-one) (Salvatore Cuzzocrea et al.); GPI 16539 (2-(4-methyl-piperazin-1-yl)-5H-benzo[c][1,5]naphthyridin-6-one) (Salvatore Cuzzocrea et al.); GPI 6150 (1,11b-dihydro-[2H]benzopyrano[4,3,2-de]isoquinolin-3-one) (Salvatore Cuzzocrea et al.); DR2313 (Calbiochem, Alexis); AG14361 (Pfizer); NU1025 (8-hydroxy-2 methyl-quinazolin-4-[3H]one) (Alexis Biochemicals); CEP 6800 (Cephalon, Pa., USA); AG 014699 (developed by collaboration among Newcastle University, Cancer Research UK, and Agouron Pharmaceuticals); ABT-888 ((2-[(R)-2-methylpyrrolidin-2-yl]-1H-benzimidazole-4-carboxamide) (Abbott Laboratories); minocycline or other tetracycline derivatives (Sigma, Alexis, et al.).

We have also used gene deletion to create PARP-1^{-/-} (homozygous) and PARP-1^{-/+} (heterozygous) mice. Observations with these mice gave consistent results, confirming that PARP inhibition is an effective mechanism for regression.

### MODES FOR CARRYING OUT THE INVENTION

Apolipoprotein E^{-/-} (ApoE^{-/-}) mice develop well-established atherosclerotic plaques when fed a high-fat diet. We used ApoE^{-/-} mice to demonstrate that the PARP inhibitor thieno[2,3-c]isoquinolin-5-one (TIQ-A), when combined with a regular diet regimen, actually induced regression of established atherosclerotic plaques, and also reduced total cholesterol and LDL. Further, plaques in the TIQ-A-treated mice had more collagen, more smooth muscle cells, displayed a thick fibrous cap, had less macrophage recruitment, and fewer foam cells. There were also decreased levels of MCP-1 and intercellular adhesion molecule (ICAM)-1, perhaps as the result of reduced TNF expression. We discovered, surprisingly, that PARP inhibition can not only help prevent the formation of atherosclerotic plaques, but that PARP inhibition also promotes the regression of existing plaques.

### Treatment of Atherosclerosis: Materials and Methods

### EXAMPLES 1-6

### Animals, Diet, and Treatment Protocols

C57BL/6 ApoE^{-/-} mice (Jackson Laboratory, Bar Harbor, Me.) were housed and bred in a pathogen-free animal care facility at LSUHSC (New Orleans, La.), and were given full access to laboratory rodent chow and water. Principles of laboratory animal care were followed (NIH publication No. 86-23, revised 1985). Experimental protocols were approved by the LSUHSC Animal Care and Use Committee. The animals were divided into six groups to provide proper controls, and to allow calculation of the significance of the results. All Groups were ApoE^{-/-} mice. The mice were fed a regular diet or a high fat diet for 12 or 16 weeks; in some cases mice that had been fed a high fat diet for 12 weeks were switched to a regular diet for the final 4 weeks (16 weeks total). Some mice were given injections of TIQ-A during the final four weeks, while others were injected with vehicle as controls:

| | | Plaque size (mm²) | | Plaque Number | |
|---|---|---|---|---|---|
| | | (median ± S.D.) | | (mean ± S.D.) | |
| | | Thoracic | Abdominal | Thoracic | Abdominal |
| Group | Diet and Treatment | Aorta | Aorta | Aorta | Aorta |
| Group 1 | Regular diet for 16 weeks, | 1.10 ± 0.15 | 0 | 2.0 ± 0.81 | 0 |
| | with vehicle injections | | | | |
| | during last 4 weeks | | | | |
| | (total of 16 weeks) | | | | |
| Group 2 | High fat diet for 16 weeks, | 12.4 ± 3.24 | 2.87 ± 0.99 | 10.3 ± 1.97 | 10.8 ± 1.07 |
| | with vehicle injections | | | | |
| | during last 4 weeks | | | | |
| | (total of 16 weeks) | | | | |
| Group 3 | High fat diet for 16 weeks, | 7.18 ± 2.02* | 3.06 ± 0.66 | 6.0 ± 1.53* | 5.7 ± 0.94* |
| | with TIQ-A injections | | | | |
| | during last 4 weeks | | | | |
| | (total of 16 weeks) | | | | |
| Group 4 | High fat diet for 12 weeks; | 12.2 ± 3.0 | 2.04 ± 0.69 | 7.0 ± 2.16 | 6.30 ± 0.94 |
| | no injections (total of 12 | | | | |
| | weeks) | | | | |
| Group 5 | High fat diet for 12 weeks, | 8.21 ± 2.87 | 3.0 ± 1.28 | 7.50 ± 1.70 | 7.50 ± 1.71 |
| | then shifted to regular diet | | | | |
| | during last 4 weeks, with | | | | |
| | vehicle injections during last | | | | |
| | 4 weeks (total of 16 weeks) | | | | |
| Group 6 | High fat diet for 12 weeks, | 5.12 ± 1.68*‡† | 1.96 ± 0.48 | 4.20 ± 0.69*‡ | 4.0 ± 1.29*‡ |
| | then shifted to regular diet | | | | |
| | during last 4 weeks, with | | | | |
| | TIQ-A injections during last | | | | |
| | 4 weeks (total of 16 weeks) | | | | |

*Statistically significant difference from Group 2 (P < 0.01)
‡Statistically significant difference from Group 5 (P < 0.01)
†Statistically significant difference from Group 3 (P < 0.01)

The high-fat diet (Harlan Teklad, Madison, Wis.) contained 21% fat by weight (0.15% cholesterol). The regular diet was normal laboratory rodent chow. Controls were given i.p. injections of vehicle three times per week, during the final four weeks. Experimental Groups received i.p. injections of the PARP inhibitor thieno[2,3-c]isoquinolin-5-one (TIQ-A) (Sigma-Aldrich, St Louis, Mo.) at the rate of 3 mg/kg, three times per week, during the last 4 weeks. At the conclusion of each protocol, the mice were fasted, anesthetized with ketamine/xylazine (60 and 3 mg/kg, respectively), and blood was drawn for serum preparation.

### EXAMPLES 7-8

### Analysis of Fasted Plasma Cholesterol and LDL

Plasma cholesterol and LDL were analyzed using a commercially available kit according to the manufacturer's instructions (Cholestech LDX, Hayward, Calif.).

### EXAMPLES 9-10

### Histology, Quantitation of Atherosclerosis, Immunohistochemistry, and Quantitation of Immunoreactivity

Perfusion-fixed aortas were dissected and prepared either for en face, Oil-Red-O staining using standard protocols, or for embedding in paraffin. Tissues were sectioned and were stained following standard protocols with haematoxylin and eosin (H&E), trichrome staining, or immunohistochemistry (IHC). Lesion areas were assessed as described in K. Oumona-Benachour et al. (2007). IHC used antibodies against murine smooth muscle actin (SMA) (Santa Cruz Biotechnologies, Santa Cruz, Calif.), or antibodies against CD68. Immunoreactivity was assessed in captured images of immunostained stained sections as described in K. Oumona-Benachour et al. (2007).

### EXAMPLE 11

### Reverse Transcription and Real-Time PCR

RNA was extracted from thoracic aortas, and cDNA was generated by standard methods. PCR Primers for MCP-1 and for actin were as described in C. Hans et al., “Thieno[2,3-c]isoquinolin-5-one (TIQ-A), a potent poly(ADP-ribose) polymerase inhibitor, promotes atherosclerotic plaque regression in high fat diet-fed ApoE-deficient mice: effects on inflammatory markers and lipid content,” J. Pharm. Exp. Ther., vol. 329, pp. 150-158 (2009, published online Jan. 5, 2009). Amplification, detection, and data analysis were performed with the iCycler real-time PCR system (Bio-Rad Laboratories, Hercules, Calif.).

Statistical analysis. Data were determined as mean±SD of values derived from groups containing at least 6 mice each. PRISM software (GraphPad, San Diego, Calif.) was used to analyze the differences between experimental groups by one-way ANOVA, followed by the Dunnett multiple-comparison test. Values of P<0.05 were considered statistically significant.

### Treatment of Atherosclerosis: Results

### EXAMPLES 12-16

### Pharmacological Inhibition of PARP by TIQ-A Promoted the Regression of Previously-established Atherosclerotic Plaques in Mice

The 12-week, high-fat diet regimen (Group 4) induced pronounced plaque formation throughout the aorta as assessed by Oil-Red-O staining. A 16-week, high-fat diet regimen (Group 2) further increased the average plaque size and number. Following the 12-week, high-fat diet regimen, switching to a regular diet during the last four weeks of the protocol (Group 5) produced a small, but statistically insignificant, reduction in plaque size and number in the thoracic region of the aorta.

Following the 12-week, high-fat diet regimen, administering TIQ-A in addition to switching to a regular diet during the last four weeks of the protocol (Group 6) resulted in statistically significant regression of established plaques in both the thoracic and abdominal regions of the aorta. There was a greater reduction in plaque number and size in the thoracic area, at the brachiocephalic region. In the abdominal region, TIQ-A induced a significant reduction in plaque number; lesion size also trended lower in the abdominal region, although the observed difference was not statistically significant.

The plaques were examined microscopically. The plaques from the mice that received a high-fat diet throughout the 16-week experiment (Group 2) were advanced, with layered lesions, multiple fibrous caps, and cholesterol-rich lipid cores within the intimal layer. They contained distinct, large, macrophage-derived foam cells and SMCs. The plaques from the Group 4 (12 weeks on high-fat diet) and Group 5 mice (12 weeks on high-fat diet, followed by 4 weeks regular diet) were similar in appearance to those from Group 2, except that they each had a thin, single-layered fibrous cap.

By contrast, microscopic examination showed the plaques to be much smaller in the mice that had been treated with TIQ-A and fed a regular diet during the final four weeks (Group 6). The plaques contained fewer foam cells, had an SMC-rich fibrous cap, and a well-contained lipid core. Trichrome staining of the Group 6 plaques clearly indicated a much thicker fibrous cap. These traits were characteristic of stable atherosclerotic plaques. Not only did PARP inhibition by TIQ-A in combination with regular diet regimen promote plaque regression but this treatment also appeared to promote plaque stability.

### EXAMPLE 17

### TIQ-A Administration Promotes Enhanced SMCs and Increased Collagen Levels in the Regressed Atherosclerotic Plaques

TIQ-A treatment produced a significant increase in smooth muscle actin immunoreactivity in the Group 6 mice. The regressed plaques were enriched in smooth muscle cells (SMCs). The SMC enrichment was accompanied by a significant increase in collagen density, as assessed by trichrome staining. These results further support the conclusion that TIQ-A not only induced plaque regression, but also promoted factors that contribute to plaque stability.

### EXAMPLE 18

### TIQ-A Treatment Induced Lower Serum Lipid Levels in the Group 6 Mice

Lowering lipid levels can also be a factor in reducing plaques. The lipid profiles from the different groups of mice, after overnight fasting, showed that TIQ-A treatment significantly lowered total cholesterol and LDL levels.

### EXAMPLES 19-21

### TIQ-A administration Reduced Macrophage Recruitment, which may be Mediated by Reduced Expression of the Chemokine MCP-1 and the Adhesion Molecule ICAM-1

Several pathways may play a role in the regression of plaques, including some that interfere with the recruitment and retention of monocytes, macrophages and foam cells, cells that appear to be required for the maintenance of advanced plaques. In particular, the continuing recruitment of macrophages into lesions is considered a major factor in maintaining the size and complexity of atherosclerotic plaques. We observed that TIQ-A treatment significantly reduced CD68 immunoreactivity in the Group 6 mice, suggesting that the population of macrophage-like cells had been reduced. Lower numbers of macrophages may affect the recruitment of new monocytes, and the egress and viability of foam cells. MCP-1 is a potent chemotactic factor that is up-regulated at sites of inflammation, and that attracts monocytes and macrophages. To confirm that the decrease in CD68-positive cells was associated with reduced expression of chemotactic factors, we used quantitative RT-PCR to monitor the effect of TIQ-A on MCP-1 expression. We measured transcription of the MCP-1 gene in the thoracic aorta using primers specific for murine MCP-1. Measured values were normalized versus measured levels of transcription of the β-actin gene. The RT-PCR data showed that expression of MCP-1 was significantly reduced following administration of TIQ-A. The reduction in MCP-1 gene expression correlated with a decrease in MCP-1 immunoreactivity of plaques in the TIQ-A-treated mice. By contrast, no difference was seen in expression of the MCP-1 receptor, CCR2.

In addition to MCP-1, the recruitment and their retention of monocytes and macrophages within atherosclerotic plaques requires the expression of adhesion molecules by endothelial and intimal cells. In particular, the adhesion molecule ICAM-1 expression is expressed in the later stages of plaques. Treatment with TIQ-A significantly reduced expression of ICAM-1 in the thoracic aortas of Group 6 mice, as assessed by quantitative RT-PCR.

### EXAMPLE 22

### TIQ-A Lowered the Expression of TNF

Other studies have shown that MCP-1 and adhesion molecules are correlated with the expression of tumor necrosis factor (TNF). We assessed TNF expression by quantitative RT-PCR in our mouse model, and found that TIQ-A significantly down-regulated TNF expression in the Group 6 mice.

### Atherosclerosis: Discussion

We have shown that PARP inhibitors such as TIQ-A can induce the regression of plaques. Inhibiting PARP may promote plaque regression through its effects on factors required to maintain advanced plaques, including adhesion molecules and inflammatory factors such as MCP-1 and TNF; reducing dyslipidemia; and inhibiting lipid-laden foam cells. Without wishing to be bound by this hypothesis, we propose that TIQ-A reduces TNF expression, which leads to reduced expression of MCP-1 and adhesion molecules, thereby producing an environment that favors foam cell death and the emigration of macrophages and foam cells from plaques.

The decreased macrophage recruitment does not appear to result from any reduced ability of macrophages to migrate to the lesion site. We observed, for example, that PARP inhibition in vitro did not affect the chemotactic response of macrophages to MCP-1 or to GM-CSF. Macrophages may respond to TIQ-A-induced reductions in chemokine expression by emigrating out of plaques. PARP inhibition not only reduces the expression of MCP-1 and adhesion molecules, but also plays a role in the death of the vascular cells that constitute atherosclerotic plaques. Indeed, while PARP inhibition protects against the death of endothelial cells and SMCs in response to a variety of inflammatory factors, including oxidized cholesterol, PARP inhibition also sensitizes lipid-laden foam cells to the cytotoxic effect of the oxidized cholesterol 7-ketocholesterol. We note that 7-ketocholesterol is an important constituent of plaques, accounting for up to 30% of the total sterols in oxidized LDL. The reduction in CD68-positive cells within plaques following TIQ-A administration may be due, in part, to sensitization to oxidized cholesterols.

### Treatment of Asthma: Materials and Methods

### EXAMPLES 23-27

### Animals, Diet, and Protocols for Sensitization and Challenge

Mice were bred in a pathogen-free facility at the Louisiana State University Health Science Center (LSUHSC), New Orleans, La., and were given unlimited access to sterilized chow and water. Animal maintenance, experimental protocols, and procedures were approved by the LSUHSC Animal Care & Use Committee. C57BL/6 mice were purchased from Jackson Laboratories (Bar Harbor, Me.). Six-week old C57BL/6 mice (n z 5) were sensitized with two, 100 μg i.p. injections of Grade V chicken ovalbumin (OVA) (Sigma-Aldrich, St. Louis Mo.), mixed with 2 mg aluminum hydroxide in saline. The two injections were given seven days apart. One week after the second sensitization the mice were placed in a Plexiglas chamber and challenged with aerosolized ovalbumin (3% OVA in saline, generated by a Bennett nebulizer, DeVilbiss, Pa.). The challenged mice were held in the chamber with the aerosolized OVA for 30 min. Additional groups of mice were treated by the same protocol, except that they received 6 mg/kg TIQ-A (i.p.) 2 hours prior to challenge, 1 hour post-challenge, or 6 hours post-challenge with OVA. Control groups were neither sensitized, nor challenged. The mice used in each experiment were from the same litter or the same family. Mice were allowed to recover, and then killed by CO₂ asphyxiation after 24 or 48 hours. The sacrificed mice were subjected to bronchio-alveolar lavage (BAL), or their lung tissues were dissected, fixed, and processed for histological analysis.

### EXAMPLES 28-31

### Organ Recovery and Staining; Th2 Cytokine and IgE Assessments

A final volume of 5 ml saline was used in BAL to assess inflammatory cells (48 hours post OVA-challenge), or 1 ml saline was used to assess cytokines or IgE (24 hours post OVA-challenge). Formalin-fixed lungs were sectioned and subjected to haematoxylin and eosin (H&E), Periodic Acid-Schiff (PAS)-staining using standard protocols. Mucin was assessed essentially as described in L. Whittaker et al., Am. J. Respir. Cell. Mol. Biol., vol. 27, pp. 593-602 (2002). Collected BAL fluids were subjected to cyto-spin, and were stained with H&E to assess inflammatory cells. Cytokines were assayed using the Bio-Rad Bioplex System for the mouse Th2 cytokines IL-4, IL-5 and IL-13, according to the manufacturer's instructions. OVA-specific IgE was quantified by a sandwich ELISA (Serotec, Raleigh, N.C.) essentially as described in Oumona et al. (2006).

### EXAMPLE 32

### Assessment of Pulmonary Function

Twenty-four hours after OVA challenge lung resistance to increasing doses of methacholine (MeCh, Sigma; 0, 25, and 50 mg/ml in isotonic saline) was assessed by forced oscillation, essentially as described in D. You et al., Respir. Res., vol. 7, pp. 107 ff (2006). Anesthetized animals were mechanically ventilated with a tidal volume of 10 ml/kg, at a frequency of 2.5 Hz, using a computer-controlled piston ventilator (FlexiVent, SCIREQ; Montreal, Canada). Just prior to data collection, the volume history of the respiratory system was standardized by inflating the lungs to measure total lung capacity. Resistance data were collected using a single-compartment model, and were normalized against baseline (i.e., resistance at 0 mg/ml MeCh).

Data analysis. All data were determined as mean±SD of values obtained from at least six mice per group, unless stated otherwise. PRISM software (GraphPad, San Diego, Calif.) was used to analyze the differences between experimental groups by one-way ANOVA followed by Dunnett's multiple comparison test.

### Treatment of Asthma: Results

### EXAMPLES 33 AND 34

### Comparison of the Effects of TIQ-A Administration Before and After Ovalbumin Challenge

Stained lung tissues examined by light microscopy showed that OVA sensitization and challenge induced clear and marked perivascular and peribronchial infiltration by eosinophils in the lungs of untreated, control mice. Administering TIQ-A prior to OVA-challenge significantly suppressed eosinophil recruitment. We also tested the efficacy of TIQ-A in controlling inflammation when administered after allergen (OVA) challenge. The infiltration of inflammatory cells into the airways was greatly reduced in animals that had received a single TIQ-A i.p. injection, either one hour or six hours after challenge. The number of eosinophils in post-challenge BAL fluids following TIQ-A administration was substantially below that seen when the drug was administered prior to challenge. These results showed not only that PARP plays a critical role in allergen-induced lung inflammation, but also that PARP inhibition after allergen exposure can reduce eosinophilia in the lungs.

### EXAMPLES 35 AND 36

### Post-challenge TIQ-A Administration Severely Suppress Th2 Cytokine and IgE Production after OVA Challenge

Important factors in allergen-induced lung inflammation include the expression of Th2-type cytokines, and the secretion of IgE. Cytokine expression promotes the generation and recruitment of eosinophils into the lung following allergen exposure. We compared the effects of PARP inhibition pre- and post-OVA challenge on the expression of IL-4, IL-5, and IL-13. Cytokine production was measured in BAL fluids taken 24 hours after challenge. Exposure to OVA induced a robust production of each of cytokines IL-4, IL-5, and IL-13. Surprisingly, these cytokines were more effectively suppressed by administering TIQ-A after the OVA challenge than before challenge. Levels of OVA-specific IgE were also measured in BAL fluid samples 24 hours post-challenge. TIQ-A caused a moderate reduction in the levels of OVA-specific IgE when administered prior to OVA challenge. Surprisingly, TIQ-A almost completely blocked IgE production when administered after OVA challenge.

### EXAMPLES 37-38

### Post-challenge TIQ-A Administration Completely Stopped Mucus Production in the Airways of OVA-challenged Mice

Excessive secretion of mucus is a symptom of allergic inflammatory responses, especially in severe and fatal asthma attacks. IL-13 has been implicated in the over-secretion of mucus during asthma attacks. We have observed that post allergen-challenge PARP inhibition suppressed IL-13 production. We investigated the effect of PARP inhibition on mucus hypersecretion in our experimental mouse model. Lung sections from different experimental groups were stained with PAS to highlight mucus-secreting goblet cells. The lungs of OVA-challenged, wild-type mice produced copious amounts of mucus, as shown by the pronounced red staining of goblet cells. By contrast, PAS-positive goblet cells were nearly absent from the lungs of OVA-challenged wild type mice that had received TIQ-A either one or six hours after challenge. Quantitative assessment of a histological mucin index showed that treatment with TIQ-A prior to OVA challenge significantly reduced mucus production. Surprisingly, post-challenge treatment with TIQ-A effectively blocked mucus production completely.

### EXAMPLE 39

### Post-challenge TIQ-A Administration Prevented Methacholine-induced Airway Hyperresponsiveness in OVA-challenged Mice

After determining that TIQ-A inhibited eosinophilia and the Th2 response, we assessed whether TIQ-A would also inhibit airway hyperresponsiveness. Lung resistance to increasing doses of methacholine was assessed as described above. Administering TIQ-A post-OVA challenge almost completely blocked airway hyperresponsiveness to inhaled methacholine (50 mg/ml) 24 hours after OVA exposure.

### Treatment of Asthma: Discussion

We have shown that inhibiting PARP after allergen exposure, for example with the PARP inhibitor TIQ-A, can reduce allergen-induced lung inflammation. The effects of TIQ-A include down-regulating the Th2 cytokines IL-4, IL-5, and IL-13, down-regulating IgE, and reducing airway hyperresponsiveness.

It was most surprising that TIQ-A was effective against allergen-induced airway eosinophilia, mucus production, and airway hyperresponsiveness when administered after allergen exposure.

### Further Trials, and Routes of Administration.

Following successful completion of animal trials, TIQ-A and other PARP inhibitors will be tested in human patients with atherosclerosis or asthma (or both) in clinical trials, conducted in compliance with applicable laws and regulations.

PARP inhibitors used in the present invention may be administered to a patient by any suitable means, including intravenous, parenteral, subcutaneous, intrapulmonary, and intranasal administration. Parenteral infusions include intramuscular, intravenous, intraarterial, or intraperitoneal administration. The compounds may also be administered transdermally, for example in the form of a slow-release subcutaneous implant. They may also be administered by inhalation.

When used to cause regression of atherosclerotic plaques, the compounds are administered after the formation of one or more such plaques in a patient, and are administered for a time and in a dosage sufficient to cause regression of one or more such existing plaques. When used to treat asthma, the compounds are administered after exposure to allergen, and may be administered before or after the onset of an asthma attack, and surprisingly are more effective when administered after the inception of a sudden-onset asthma attack.

Pharmaceutically acceptable carrier preparations include sterile, aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. The active therapeutic ingredient may be mixed with excipients that are pharmaceutically acceptable and are compatible with the active ingredient. Suitable excipients include water, saline, dextrose, glycerol and ethanol, or combinations thereof. Intravenous vehicles include fluid and nutrient replenishers, electrolyte replenishers, such as those based on Ringer's dextrose, and the like. Preservatives and other additives may also be present such as, for example, antimicrobials, anti-oxidants, chelating agents, inert gases, and the like.

The form may vary depending upon the route of administration. For example, compositions for injection or inhalation may be provided in the form of an ampoule, each containing a unit dose amount, or in the form of a container containing multiple doses.

A compound in accordance with the present invention may be formulated into therapeutic compositions as pharmaceutically acceptable salts. These salts include acid addition salts formed with inorganic acids, for example hydrochloric or phosphoric acid, or organic acids such as acetic, oxalic, or tartaric acid, and the like. Salts also include those formed from inorganic bases such as, for example, sodium, potassium, ammonium, calcium or ferric hydroxides, and organic bases such as isopropylamine, trimethylamine, histidine, procaine and the like.

A method for controlling the duration of action comprises incorporating the active compound into particles of a polymeric substance such as a polyester, peptide, hydrogel, polylactide/glycolide copolymer, or ethylenevinylacetate copolymers. Alternatively, an active compound may be encapsulated in microcapsules prepared, for example, by coacervation techniques or by interfacial polymerization, for example, by the use of hydroxymethylcellulose or gelatin-microcapsules or poly(methylmethacrylate) microcapsules, respectively, or in a colloid drug delivery system. Colloidal dispersion systems include macromolecule complexes, nanocapsules, microspheres, beads, and lipid-based systems including oil-in-water emulsions, micelles, mixed micelles, and liposomes.

As used in the specification and claims, an “effective amount” or an “effective dosage” of a compound is an amount or dosage, that when administered to a patient (whether as a single dose or as a time course of treatment) causes the regression of one or more existing atherosclerotic plaques to a clinically significant degree, or that relieves one or more symptoms of a sudden-onset asthma attack to a clinically significant degree; or alternatively, to a statistically significant degree as compared to control. “Statistical significance” means significance at the P<0.05 level, or such other measure of statistical significance as would be used by those of skill in the art of biomedical statistics in the context of a particular type of treatment or prophylaxis.

As used in the specification and claims, the “assaying” of atherosclerotic plaques may be carried out in any manner that is known in the art or that is developed in the future. The term “assaying” refers generally to a method for diagnosing the presence, size, or grade of one or more atherosclerotic plaques. Such “assaying” may optionally involve biopsy of a plaque, but it need not necessarily employ biopsy. Methods other than biopsy for carrying out assays include, for example, coronary angiography, computerized tomography (CT), including coronary calcium scoring by CT, magnetic resonance imaging, transesophageal echocardiography, or observation of one or more arteries by ultrasound or intravascular ultrasound.

The complete disclosures of all references cited in this specification, including the priority application and all its appendices, are hereby incorporated by reference. Also incorporated by reference are the complete disclosures of the following works by us and our colleagues: C. Hans et al., “Poly(ADP-ribose) Polymerase-1 Inhibition Prevents High Fat Diet-induced Cardiac Hypertrophy by Modulating Matrix-destabilizing Proteases and Oxidative Stress,” Circulation, vol. 118, p. S_{—}282-c (Abstract 311) (2008); C. Hans et al., “Differential Roles of PARP-1 in oxidants-mediated cell death in an in vitro model mimicking atherosclerotic plaque dynamics: implication in atherosclerosis,” FASEB J., vol. 21, p. A445-b (2007); C. Hans et al., “PARP-1 inhibition, pharmacologically or genetically, reduces plaque size and promotes factors of plaque stability in an ApoE−/− mouse model of atherosclerosis,” FASEB J., vol. 20, p. LB106-d-107 (2006); C. Hans et al., “Thieno[2,3-c]isoquinolin-5-one (TIQ-A), a Potent Poly(ADP-ribose) Polymerase Inhibitor, Promotes Atherosclerotic Plaque Regression in High Fat Diet-fed ApoE-deficient Mice: Effects on Inflammatory Markers and Lipid Content,” Circulation, vol. 118, p. S_{—}369-a, (Abstract 1685) (2008); C. Hans et al., “Differential effects of PARP inhibition on vascular cell survival and ACAT-1 expression favouring atherosclerotic plaque stability,” Cardiovasc. Res., vol. 78, pp. 429-439 (2008); A. Naura et al., “Reciprocal regulation of iNOS and PARP-1 during allergen-induced eosinophilia,” Eur Respir J. vol. 33, pp. 252-262 (2009, published online Oct. 1, 2008); A. Naura et al., “Post-allergen challenge inhibition of poly(ADP-ribose) polymerase harbors therapeutic potential for treatment of allergic airway inflammation,” Clin. Exp. Allergy, vol. 38, pp. 839-846, 2008. In the event of an otherwise irreconcilable conflict, however, the present specification shall control.

What is claimed:

1. A method for relieving symptoms of a sudden-onset asthma attack in a human; said method comprising administering an effective amount of a PARP inhibitor to a human who is experiencing an acute asthma attack, wherein said PARP inhibitor is administered from 0 hours to 12 hours after the beginning of the sudden-onset asthma attack.

2. The method of claim 1, wherein the PARP inhibitor is administered from 0 hours to 6 hours after the beginning of the sudden-onset asthma attack.

3. The method of claim 1, wherein the PARP inhibitor is administered from 0 hours to 1 hours after the beginning of the sudden-onset asthma attack.

4. The method of claim 1, wherein the PARP inhibitor is selected from the group consisting of TIQ-A; AIQ; 3-AB; PJ-34; 1,5-Isoquinolinediol; 3-Methyl-5-AIQ hydrochloride; 4-Amino-1,8-naphthalimide; 4-Hydroxyquinazoline; 5-AIQ hydrochloride; 5-Iodo-6-amino-1,2-benzopyrone; 6(5H)-Phenanthridinone; EB-47 dihydrochloride dihydrate; NU1025; DR2313; BSI 401; BSI 201; AZD 2281; INO 1001; GPI 15427; GPI 16539; GPI 6150; DR2313; AG14361; NU1025; CEP 6800; AG 014699; ABT-888; minocycline; tetracycline; and derivatives of these compounds.

5. The method of claim 1, wherein the PARP inhibitor is TIQ-A.

6. The method of claim 1, wherein the PARP inhibitor is a PARP-1 inhibitor.

7. A method for inducing the regression of one or more existing atherosclerotic plaques in a human; said method comprising administering an effective amount of a PARP inhibitor over time to a human who has previously been diagnosed with one or more atherosclerotic plaques; then assaying one or more of the plaques to confirm whether one or more of the existing plaques has regressed in response to the PARP inhibitor; and then, if said assaying step indicates that one or more of the plaques has regressed, continuing further administration of the PARP inhibitor for an additional time to induce further regression of one or more of the plaques.

8. The method of claim 7, wherein the PARP inhibitor is selected from the group consisting of TIQ-A; AIQ; 3-AB; PJ-34; 1,5-Isoquinolinediol; 3-Methyl-5-AIQ hydrochloride; 4-Amino-1,8-naphthalimide; 4-Hydroxyquinazoline; 5-AIQ hydrochloride; 5-Iodo-6-amino-1,2-benzopyrone; 6(5H)-Phenanthridinone; EB-47 dihydrochloride dihydrate; NU1025; DR2313; BSI 401; BSI 201; AZD 2281; INO 1001; GPI 15427; GPI 16539; GPI 6150; DR2313; AG14361; NU1025; CEP 6800; AG 014699; ABT-888; minocycline; tetracycline; and derivatives of these compounds.

9. The method of claim 7, wherein the PARP inhibitor is TIQ-A.

10. The method of claim 7, wherein the PARP inhibitor is a PARP-1 inhibitor.
